(19) United States
(12) Patent Application Publication
(10) Pub. No.: US 2013/0158471 A1
(43) Pub. Date: Jun. 20, 2013

(54) Dual Cannula System for Enteric Feeding

(71) Applicants: James V. Neel, Santa Rosa, CA (US); Matthew J. Birdsall, Santa Rosa, CA (US); Richard L. Klein, Santa Rosa, CA (US)

(72) Inventors: James V. Neel, Santa Rosa, CA (US); Matthew J. Birdsall, Santa Rosa, CA (US); Richard L. Klein, Santa Rosa, CA (US)

(21) Appl. No.: 13/328,971

(22) Filed: Dec. 16, 2011

(51) Int. Cl. A61M 25/14; A61M 1/00

(52) U.S. Cl. 604/35; 604/264; 604/516

(57) ABSTRACT

A dual cannula apparatus is provided for insertion into the gastrointestinal tract. One end of a first outer cannula is placed within the small intestine providing a guide for advancement of a second inner cannula. The second inner cannula extends beyond the first outer cannula and provides a source of nutritional support and pharmacological therapy. The first outer cannula has a radiopaque tip to facilitate placement thereof under x-ray or fluoroscopic guidance. The first outer cannula can also be provided with a flared end for maintenance with the naris. The second inner cannula includes one or more openings for flow of the nutritional support or pharmacological therapy into the small intestine.

### BACKGROUND OF THE INVENTION

The advent of nasoenteric feeding tubes has improved the ability of care givers to provide nutritional support, fluids, medicines, and other therapeutic agents to patients who are debilitated, comatose or otherwise in a condition requiring placement of such feeding tubes. These tubes are typically made of soft, pliable polymeric material such as polyurethane and are well tolerated by the patient. The tubes are typically weighted at their distal end with materials such as mercury or tungsten, and can be positioned using a fluoroscopic procedure, within the small intestine or bowel to reduce the risk of vomiting and aspiration. Positioning can also occur passively with confirmation using serial x-ray evaluation. The weighting of the distal end of the tubes helps to facilitate their insertion and positioning. The distal end has one or more apertures or ports to allow the flow of fluids, medicines, nutrients there through. Nasoenteric feeding is preferred over intravenous feeding because it helps to maintain integrity of the intestinal tract, and it generally provides more complete nutrition.

Inadvertent dislodgment of the feeding tube, however, presents a frustrating, and potentially dangerous, situation. Displacement of the feeding tube can occur in a variety of ways such as: a confused patient accidentally or intentionally dislodging it; or accidentally by hospital or other nursing personnel. Unfortunately, such inadvertent dislodgment is a fairly frequent occurrence. And such displacement can lead to aspiration of the nutritional support into the lungs, causing pneumonia, respiratory complications, and, possibly, death.

Given the degree of occurrence, physicians and nurses have tried various methods to prevent patients from dislodging the feeding tube such as taping or applying adhesive bandage to secure the proximal end of the tube to the patient's nose or, alternatively, using wrist restraints to prevent the patient from accessing the area around the patient's nose, or any combination thereof. The taping, however, can lead to other issues such as skin irritation and breakdown, and there remain a significant number of patients who still dislodge the tubes despite the use of these methods to prevent the dislodgment.

Furthermore, during placement, these tubes do not always pass on their own accord into the small bowel. As mentioned above, they can be actively placed under fluoroscopy or passively, if a patient is in an intensive care setting and not moveable, using serial x-rays to determine when the distal end of the feeding tube is properly positioned in the small bowel. With passive placement of conventional single lumen feed tubes, a guide wire or stylet is typically inserted into the tube and the tube and wire are inserted into the stomach. The patient is usually thereafter given a gastrointestinal stimulant such as Reglan®, to increase gastric motility to encourage passage of the feeding tube into the small intestine. To confirm proper placement, serial x-rays are taken until placement in the small intestine is confirmed. If the feeding tube does not pass into the small bowel or if there is some urgency to achieve proper tube placement, the feeding tube is placed by a radiologist. The radiologist uses fluoroscopic guidance to ensure proper placement in the small intestine. The cost of this procedure is not insignificant and must be repeated if or when a feeding tube is displaced.

Moreover, these feeding tubes can become clogged—either with nutritional supplements or medications, particularly pills which are ground prior to passing them through the feed tube. And if attempts at flushing to clear the tube obstruction are unsuccessful, the tube needs to be removed and replaced and its repositioning verified using one or more of the aforementioned strategies involving passive or active placement and utilization of serial x-ray and, possibly, additional fluoroscopic evaluation.

### BRIEF SUMMARY OF THE INVENTION

It is an objective of the present invention to overcome the failings of conventional feeding tube devices with their attendant increased heath care costs represented by replacement after such dislodging or the treatment of associated complications such as aspiration and pneumonia and the frustration experienced by care givers with such displacement. The present invention addresses this shortcoming by minimizing instances of dislodgment of such devices.

It is a further advantage of the invention that its design significantly reduces the need to replace the feeding system due to clogging of the tube providing sustenance and medication.

Moreover, the invention should also reduce interventions used by care givers to prevent displacement of such feeding tubes. These practices include taping the tube to the patient's nose or restraining the patient's wrists—neither of which is desirable.

The present invention addresses these aforementioned shortcomings of conventional feeding tubes and is useful in both a nasogastric application, where the feeding tube is inserted nasally and extends down the esophagus through the stomach and into the small intestine, and or in a percutaneous endoscopic gastronomy application where the feeding tube is placed through the abdominal wall directly into the stomach and extending into the small intestine.

The invention presents a coaxial tube or dual cannula design. In the nasogastric enteric feeding application, the outer tube extends from the nose to the small intestine. The proximal portion of the outer cannula is nested in the naris. By such placement, the patient would have much more difficulty grabbing and pulling on the outer cannula, minimizing the risk of dislodging the outer cannula and its distal end's position within the small intestine.

In a preferred embodiment, the inner tube or cannula, is threaded or passed through the outer cannula until its distal end extends beyond the outer cannula. Although it is understood that since the outer tube is placed within the small bowel, that the inner tube need not necessarily extend beyond the outer tube. In this manner, the inner tube, which carries the nutritional support, fluids, therapeutic agents, and the like, could be intentionally or accidentally pulled by the patient without disrupting the position of the outer cannula. With this system of dual inner and outer tubes, whether the inner tube is partially dislodged or needs to be replaced by the nurse due to clogging, for example, it could be replaced or reinserted using the outer cannula as a guide to ensure proper placement. Also, if the inner cannula is accidentally or intentionally dislodged by the patient, the outer tube would prevent aspiration. With this dual cannula system, the need for subsequent radiologic intervention, and its associated costs, is significantly reduced if not eliminated.

### BRIEF DESCRIPTION OF THE DRAWINGS

FIG. 1 is a perspective view of the dual catheter system constructed in accordance with an embodiment of the invention.

FIG. 2A is an enlarged view of the proximal end of a first outer cannula constructed in accordance with an embodiment of the invention.

FIG. 2B is an enlarged view of the proximal end of a first outer cannula constructed in accordance with an alternate embodiment of the invention.

FIG. 3A is a cross section taken along line 3A-3A in FIG. 1.

FIG. 3B is a cross section taken along line 3A-3A in FIG. 1 of an alternate embodiment of the invention.

FIG. 3C is a cross section taken along line 3A-3A in FIG. 1, with the second inner cannula removed, of an embodiment of the construction of the first outer cannula.

FIGS. 4A, 4B and 4C are enlarged views representing various embodiments of the distal portion of a second inner cannula.

FIG. 4D is a cross section taken along line 4D-4D of FIG. 4A of an alternate embodiment of a second inner cannula.

FIG. 5 is a perspective of a human patient depicting a dual cannula system constructed in accordance with the invention and positioned within the gastrointestinal tract through the nose.

FIG. 6 is a perspective of a human patient depicting a dual cannula system constructed in accordance with the invention and positioned within the gastrointestinal tract through percutaneous gastronomy.

FIG. 7A is a top plan view of the anchor portion of the embodiment shown in FIG. 6.

FIG. 7B is a sectional view taken along line 7B-7B of the anchor shown in FIG. 7A.

### DETAILED DESCRIPTION OF THE INVENTION

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present invention

The terms “a,” “an,” “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Moreover, and more particularly, terms such as “sheath,” “catheter,” “tube” or “cannula,” for the purposes of describing elements of the invention, are intended to be construed as interchangeable. Terms such as “ports” or “openings” are intended to be similarly construed. In addition, the terms “guidewire” and “stylet” are also intended to be construed as interchangeable. Similarly, the terms “threaded,” “tracked” and “passed” are intended to be construed as interchangeable.

The invention presents a coaxial tube or, expressed another way, a dual cannula design. In an application for nasogastric enteric feeding, the outer tube would extend from the nose to the small intestine. The proximal portion of the outer cannula would be nested in the naris. With such placement, the patient would have greater difficulty grabbing and pulling on the outer cannula, minimizing the risk of dislodging the outer cannula and its distal end's position within the small intestine. In an application for percutaneous endoscopic gastric feeding, the outer sheath would extend from a puncture site on the abdominal wall, through the stomach and into the small intestine.

The inner tube or cannula, which carries the nutritional support, fluids, therapeutic agents, and the like, could be intentionally or accidentally pulled by the patient or caregiver without disrupting the position of the outer cannula. With this system of dual inner and outer tubes, whether the inner tube is partially dislodged or needs to be replaced by the nurse due to clogging, for example, it could be replaced or reinserted using the outer cannula as a guide to ensure proper repositioning. Also, if the inner cannula is accidentally or intentionally dislodged by the patient, the outer tube would prevent aspiration. With this dual cannula system, the need for subsequent radiologic intervention, and its associated costs, is significantly reduced if not eliminated.

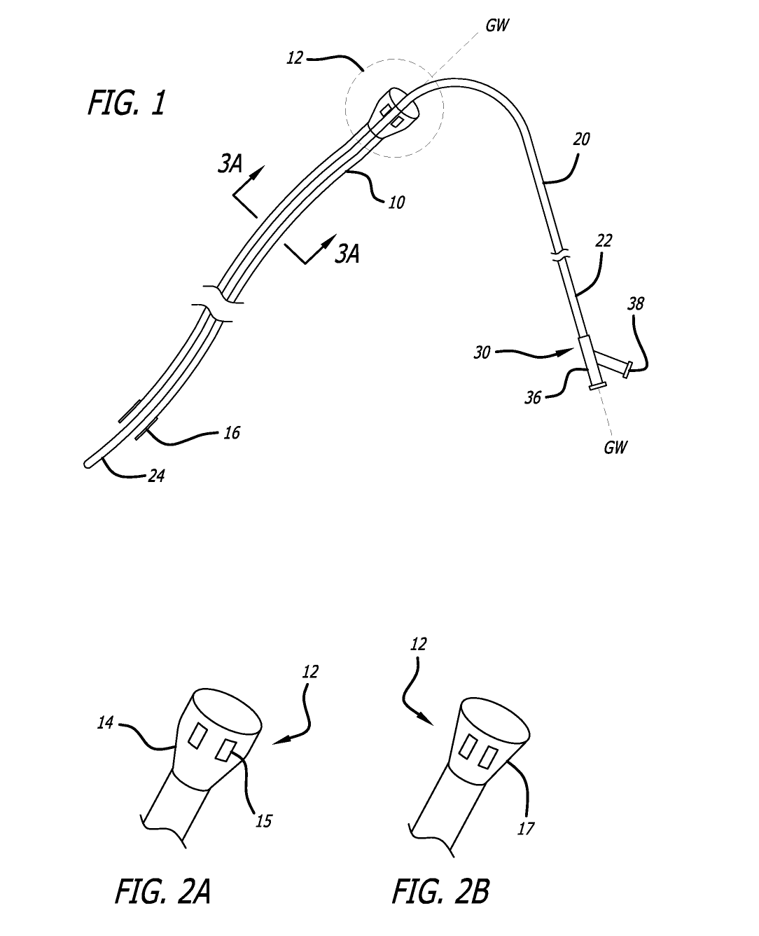

Referring with more specificity to the figures, FIG. 1 illustrates a dual catheter system constructed in accordance with an embodiment of the invention. A first outer sheath or cannula 10 is hollow, having a proximal end generally indicated at 12, a distal end 16 and an inner and outer diameter. The distal tip or end 16 is preferably radiopaque. Radiopacity of distal end 16 can be provided, for example, by impregnating or incorporating a radiopaque material such as barium, iodine, tungsten, tantalum, gold, platinum or iridium into the distal tip 16 or by affixing, such as by adhesive or swaging, a radiopaque marker band to the outer surface of distal end 16. The marker band can be fabricated from radiopaque materials such as gold, tantalum, platinum, iridium, platinum-iridium alloy, or other suitable materials and alloys.

A second inner catheter 20 is hollow and has a proximal end 22 and a distal end generally indicated at 24. Second inner cannula 20 has an outer diameter that is less than the inner diameter of first outer sheath 10. Second inner cannula 20 is preferably longer than first outer sheath 10. Second inner cannula 20 is preferably soft or pliant and fabricated from a material from a group including polyurethane, polyvinyl chloride, polyethylene, polyethyleneterephalate or other suitable polymeric materials. Second inner cannula 20 can also include indicators on its outside surface that are visible both to the care provider and visible under x-ray or fluoroscopy. Such indicators can be in the form of bands, ink, laser markings or the like. Such markings placed on the distal end of second inner cannula 20 would confirm that distal end 24 extends beyond distal tip 16. Similarly, such markings placed on proximal end 22 of second inner cannula 20 would confirm that distal end 24 extends beyond distal tip 16 and by how far it is extending beyond distal tip 16 and into the small intestine. In this manner, the care giver knows more precisely when an inner cannula is properly repositioned after dislodgement.

Second inner cannula 20 is preferably longer than first outer sheath 10. Since first outer sheath 10 provides a sealed conduit from the nose to the small intestine, however, second inner cannula 20 need not be longer than first outer sheath 10, As long as distal end 24 is within first outer sheath 10 and extends from the patient's nose to hub 30, its length is sufficient.

Turning to FIGS. 2A and 2B, the proximal end portion 12 of first outer cannula 10 is shown in more detail. This proximal end portion is preferably either flared, as indicated at 14 in FIG. 2A, or frustoconical, as indicated at 17 in FIG. 2B. Either embodiment may be provided with one or more holes 15 to allow for the drawing of air there through. The proximal end portion 12 is preferable made from a soft rubber, silicone, urethane, foam or the like.

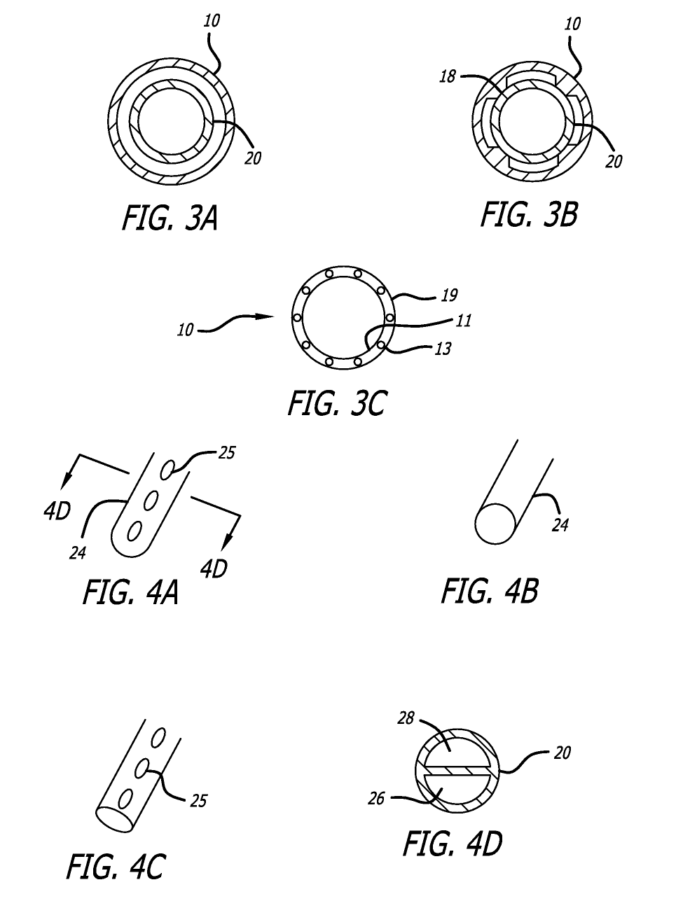

FIGS. 3A shows the relationship of the dual sheath system when the second inner catheter 20 is threaded or passed into the first outer catheter 10. FIG. 3B is an alternate embodiment of FIG. 3A showing a plurality of ridges 18 formed longitudinally along the inner wall of first outer sheath 10. In this embodiment shown in

FIG. 3B, the amount of contact between the two sheaths is decreased thereby reducing the amount of friction there between.

FIG. 3C indicates one preferred construction of first outer sheath 10. In this embodiment, first outer sheath 10 is formed using a tri-layer, braid reinforced extrusion process. More particularly, first outer sheath 10 has an inner wall 11, intermediate layer 13 and outer wall 19. In this embodiment, inner wall 11 is formed of a low-friction material such as high density polyethylene (HDPE) polymer, possibly incorporating surface texturing. The braid reinforcing layer can be formed of metal, such as stainless steel, or Kevlar, for example. Outer layer 19 is formed of a block co-polymer such as PEBAX. The braided layer provides axial or columnar strength and kink resistance. The HDPE inner wall provides lubricity thereby minimizing friction between the inner wall 11 and the second inner catheter 20. While it is preferable to have first outer sheath 10 be a tri-layer extruded braided material, any suitable material may be used to fabricate the first outer sheath 10 so long as it provides sufficient resistance to kinking and sufficient columnar strength. For example, first outer sheath 10 could be formed of a braided monolayer extrusion, or it could be of a bi-layer or tri-layer non-braided construction. Again, as long as sufficient resistance to kinking and sufficient axial strength are provided. Other polymers that could be employed in the construction of first outer sheath 10 include polyethylene (PE), polyurethane, or polytetrafluoroethylene (PTFE).

Alternate embodiments of the distal portion 24 of second inner cannula 20 are depicted in FIGS. 4A, 4B and 4C. In FIG. 4A, distal end 24 is close ended and has a plurality of ports 25. FIG. 4B indicates the distal portion to be open ended. While FIG. 4C shows a distal portion that is both open ended and having a plurality of openings 25. Any of these embodiments provide for flow of materials, liquids or gases through the second inner catheter 20, exiting from its distal end 24.

FIG. 4D suggests an embodiment where second inner sheath 20 is a dual lumen. As shown in FIG. 1, the proximal end 22 of second inner sheath 20 terminates with a fitting or luer as indicated at 30. The fitting has two channels, 36 which communicates with lumen 26 for the introduction of feeding materials, nutritional supplements, therapeutic agents and the like, and 38 which communicates with lumen 28 to apply a suction In this embodiment, first lumen 26 would communicate with ports or openings in the distal end 24, while second lumen 28 would communicate with a vent (not shown) to allow aspiration.

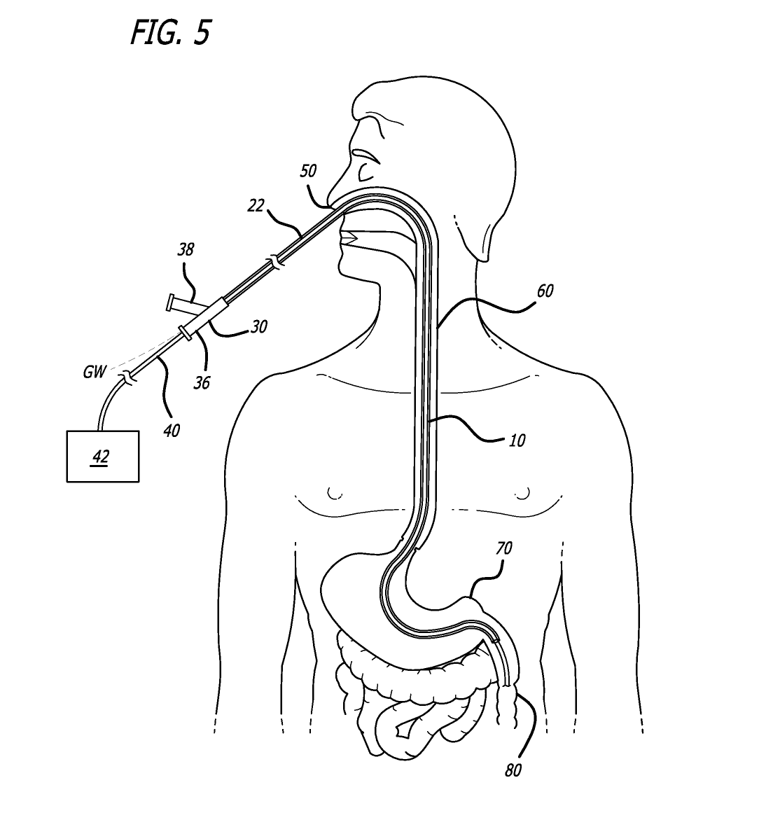

The dual cannula system described above will now be explained with regard to a particular application. FIG. 5 shows the coaxial catheter system constructed in accordance with the present invention and inserted nasally into a patient's gastrointestinal tract. More specifically, outer catheter 10 is first inserted through a patient's nostril 50 extending through the patient's esophagus 60, stomach 70 and into the small intestine 80. Radiopaque tip 16 is used as a landmark to determine whether the distal end is positioned in the small intestine. Once the first outer sheath 10 is positioned in the small intestine, it is used as a guide for passing the second inner sheath 20 through the first outer sheath and into the small intestine 80. Distal portion 16 of outer catheter 10 is preferably a soft, atraumatic tip to ease movement of the outer catheter through the gastrointestinal tract and minimize injury or aggravation of the gastrointestinal tract during delivery.

Preferably, first outer sheath 10 is placed such that it extends three inches to nine inches into the small intestine and second inner sheath 20 extends approximately two inches to six inches beyond radiopaque tip 16 to allow full exposure of the openings in its distal end 24 to the small intestine 80. A guidewire or stylet (not shown) may be used with either the outer sheath or inner sheath to provide additional columnar support thereby facilitating placement of one or both sheaths. Flared proximal end 14, of first outer sheath 10, is intended to seat within the patient's naris and maintain position therein. Luer 30 is connected to a feed pump assembly by external tube 40. Alternatively, passage 36 may be used to deliver therapeutic agents directly into the small intestine.

In the embodiment shown and described with reference to FIG. 5, it is understood that the outer sheath will have sufficient length to extend from the naris to a position within the small bowel and that the inner sheath will have a sufficient length to extend from luer 30 and beyond the distal end of the outer sheath and into the small bowel. It is also understood that either or both sheaths will be sized depending on whether it is for adult or pediatric use. It is also understood that since the outer sheath is already placed within the small bowel, that the inner sheath need not necessarily extend past the distal end of the outer sheath. Also, the dual cannula system can alternately be placed by first allowing a weighted inner sheath to be advanced into the small bowel by gastric motility. The outer sheath is then advanced over the inner tube until its distal tip is placed with the small intestine.

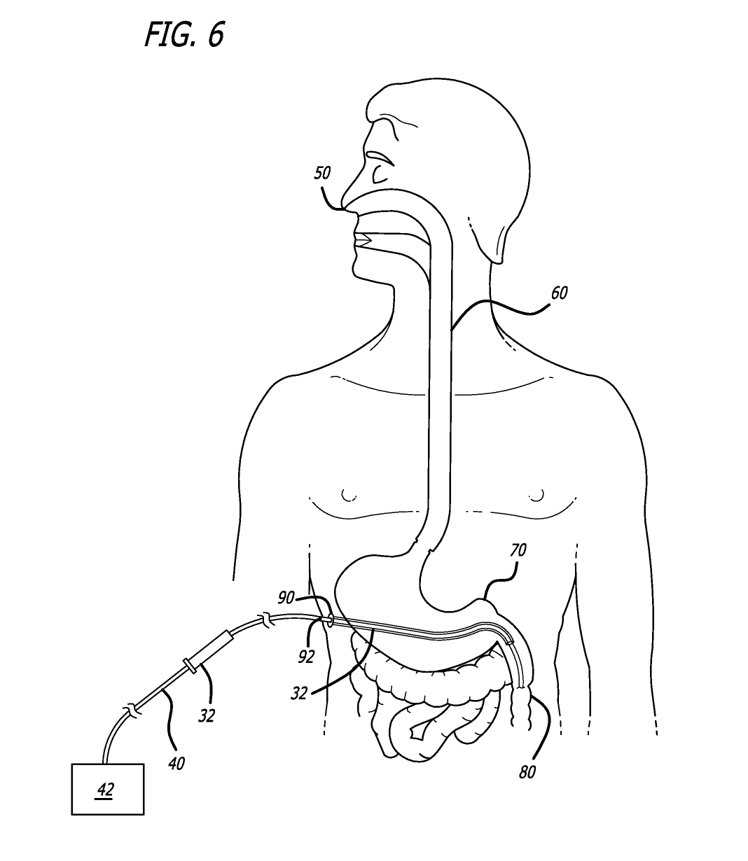

The coaxial sheath system described above will now be explained with regard to a second particular application. FIG. 6 shows the coaxial catheter system constructed in accordance with the present invention and inserted through a puncture, made mid-torso, into a patient's gastrointestinal tract. More specifically, outer catheter 10 is first inserted through an anchor 90 placed at the site of the puncture, extending through the patient's stomach 70 and into the small intestine 80. Radiopaque tip 16 is used as a landmark to determine whether the distal end is positioned in the small intestine. Once the first outer sheath 10 is positioned in the small intestine, it is used as a guide for passing the second inner sheath 20 through the first outer sheath and into the small intestine 80.

Preferably, second inner sheath 20 extends approximately two inches to six inches beyond radiopaque tip 16 to allow full exposure of the openings in its distal end 24 to the small intestine 80. A guidewire or stylet (not shown) may be used with either the outer sheath or inner sheath to provide additional columnar support thereby facilitating placement of one or both sheaths.

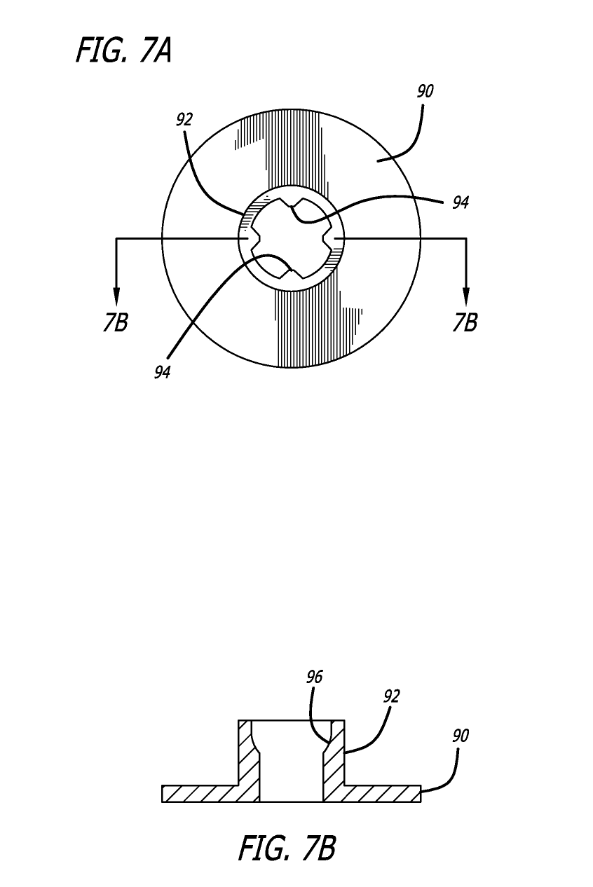

As shown in FIGS. 7A and 7B, anchor 90 includes boss 92 having plurality of elastomeric flaps 94. Flared proximal end 14, of first outer sheath 10, is adapted to seat matching inner surface 96 of boss 92. Elastomeric flaps 94 maintain flared proximal end 14 within anchor 90.

Returning to FIG. 6, luer 32 is connected to a feed pump assembly 42 by external tube 40. In the embodiment of FIG. 6, the luer is depicted as a single passageway fitting. With the present invention, risk of materials being passed into the stomach, as opposed to the small intestine when the second inner sheath is dislodge are minimized, if not eliminated, due to the presence of the first outer sheath. Therefore, the need for a dual luer with a second fitting for suction may be alleviated. It is contemplated by the present invention that a single luer fitting or a dual luer fitting may be used with the embodiments of FIGS. 5 and 6.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this invention are described herein, including the best mode known to the inventors for carrying out the invention. Of course, variations on these described embodiments will become apparent to those of ordinary skill in the art upon reading the foregoing description. For example, nasoenteric feeding tubes described in the Background of the Invention above could be substituted for the second inner catheter. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Specific embodiments disclosed herein may be further limited in the claims using consisting of or consisting essentially of language. When used in the claims, whether as filed or added per amendment, the transition term “consisting of” excludes any element, step, or ingredient not specified in the claims. The transition term “consisting essentially of” limits the scope of a claim to the specified materials or steps and those that do not materially affect the basic and novel characteristic(s). Embodiments of the invention so claimed are inherently or expressly described and enabled herein.

In closing, it is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

What is claimed:

1. A dual sheath system for placement within a conduit of a body comprising:
a first outer sheath having a proximal end and a distal end, an outer diameter and an inner diameter, and an outer wall and an inner wall;
a second inner sheath having a length that is longer than said first outer sheath, a proximal end and a distal end, one or more ports located proximate the distal end of said second inner sheath, and an outer diameter that is less than the inner diameter of the of said first outer sheath;
wherein said second inner sheath is independently moveable in a coaxial relationship with and within said first outer sheath such that said one or more ports extend distally of said distal end of said first outer sheath, and said proximal end of second inner sheath extends proximally of said proximal end of said first outer sheath.

2. The dual sheath system according to claim 1 wherein said first outer sheath has a radiopaque area at its distal end.

3. The dual sheath system according to claim 2 wherein the radiopaque area comprises impregnating the distal end of said first outer sheath with a radiopaque material.

4. The dual sheath system according to claim 2 wherein the radiopaque area comprises a radiopaque band secured to the outer wall of said first outer sheath.

5. The dual sheath system according to claim 1 wherein the inner wall of said first outer sheath further comprises a plurality of ridges extending along the inner wall for engaging said second inner sheath and reducing friction between said first outer sheath and said second inner sheath when said second inner sheath is independently moved within and in relation to said first outer sheath.

6. The dual sheath system according to claim 1 wherein said first outer sheath is extruded from a braid reinforced monolayer material.

7. The dual sheath system according to claim 1 wherein said first outer sheath is extruded from a non-braided multilayer material.

8. The dual sheath system according to claim 1 wherein said first outer sheath is extruded from a trilayer material having an inner layer, an intermediate layer, and an outer layer and wherein said intermediate layer is braid reinforced.

9. The dual sheath system according to claim 1 wherein said second inner sheath further comprises one or more markings to indicate whether said one or more ports are extending distally of said distal end of said first outer sheath.

10. The dual sheath system according to claim 1 wherein the dual sheath system is inserted within the gastrointestinal tract of a patient.

11. An apparatus for enteric feeding of a patient comprising:
a first catheter extending from proximate the patient's naris through the patient's esophagus, stomach and into a position proximate the patient's small intestine; and
a second catheter slideably disposed with said first catheter.

12. The apparatus according to claim 11 wherein said second catheter is longer than said first catheter and moveable to a position distal of said first catheter within the patient's small intestine.

13. The apparatus according to claim 11 wherein said first catheter further comprises:
a proximal end and a distal end;
said proximal end of said first catheter is placed in a position within the patient's naris; and
said distal end of said first catheter has a radiopaque region to assist placement of said distal end within the patient's small intestine.

14. The apparatus according to claim 13 wherein the first catheter further comprises a flared proximal end to facilitate and maintain seating of the flared proximal end within the patient's naris.

15. The apparatus according to claim 14 wherein said flared proximal end is made from soft rubber or a spongy material.

16. The apparatus according to claim 14 wherein the flared proximal end of said first catheter further comprises at least one hole to allow the patient to draw air there through.

17. The apparatus according to claim 11 wherein said first catheter is substantially kink resistant.

18. The apparatus according to claim 12 wherein said second catheter further comprises a proximal end and a distal end, wherein when said distal end of said second catheter is moved to a position distal of said first catheter and into the patient's small intestine, and the proximal end of said second catheter extends from the patient's naris.

19. The apparatus according to claim 18 wherein the proximal end of said second catheter further comprises a fitting for connecting said second catheter to a supply of nutrition, fluids, medicines or other therapeutic agents.

20. The apparatus according to claim 18 wherein said distal end of said second catheter further comprises one or more ports for delivery of a supply of nutrition, fluids, medicines or other therapeutic agents to the patient's small intestine.

21. The apparatus according to claim 14 wherein said second catheter further comprises a radiopaque marker at its distal end to indicate when the distal end of said second catheter has exited the distal end of said first catheter and entered the patient's small intestine.

22. The apparatus according to claim 14 wherein said second catheter further comprises a visible marker proximate the proximal end of said second catheter to indicate when the distal end of said second catheter has exited the distal end of said first catheter and entered the patient's small intestine.

23. The apparatus according to claim 19 wherein said second catheter has a feeding passage and a suction passage and the fitting comprises a dual luer having one passageway that communicates with the feeding passage and a second passageway that communicates with the suction passage.

24. A method for enteric feeding of a patient comprising the steps of:
providing a catheter apparatus having a first outer cannula having a length and first and second ends, and a second inner cannula with first and second ends, at least one exit port proximate the first end, and a length that is longer than the length of the first outer cannula;
advancing said first outer cannula such that the first end of said first outer cannula is in a position within the patient's small intestine;
advancing said second inner cannula through said first outer cannula to a position proximate the first end of said first outer cannula such that the second end of said second inner cannula extends beyond the second end of said first outer cannula.

25. The method recited in claim 24 further comprising the step of placing a guidewire into said first outer cannula prior to advancing it into a patient's small intestine to provide columnar support to facilitate tracking said first outer cannula into the small intestine of a patient.

26. The method recited in claim 24 wherein the first end of said first outer cannula is provided with a radiopaque tip and said first outer cannula is advanced under serial x-ray or fluoroscopic guidance until it is determined to have entered the small intestine of a patient.

27. The method recited in claim 24 further comprises the step of advancing said second inner cannula such that the first end of said second inner cannula extends beyond the first end of said first outer cannula.

28. The method recited in claim 27 further comprising the step of placing a guidewire into said second cannula prior to advancing it through said first outer cannula to provide columnar support and facilitate its transmission through said first outer cannula and into the small intestine of a patient.

29. The method recited in claim 24 further comprising the step of introducing a supply of nutrients, fluids, medicines or other therapeutic agents into the second end of said second inner cannula such that said supply flows through said second inner cannula, out the at least on exit port and into the small intestine of a patient.

30. The method recited in claim 27 further comprising the step of introducing a supply of nutrients, fluids, medicines or other therapeutic agents into the second end of said second inner cannula such that said supply flows through said second inner cannula, out the at least on exit port and into the small intestine of a patient.

31. The method recited in claim 24 further comprising the step of connecting an external tube between the second end of said second inner cannula and a feed pump assembly.

32. The method recited in claim 24 further comprising the steps of:
inserting said first outer cannula into a patient's nose; and
advancing said first outer cannula along the esophagus, through the stomach and into the small intestine.

33. The method recited in claim 24 further comprising the steps of:
making a puncture mid-torso of a patient proximate the stomach;
affixing an anchor at the site of the puncture;
inserting said first outer cannula percutaneously through the anchor and into the stomach; and
advancing said first outer cannula through the stomach and into the small intestine.
